SEQUENCE LISTING (18 sequences)

SEQ ID NO: 1 - DNA, 21 bp, Artificial
  Forward Primer
ccccagattttgagcactacc

SEQ ID NO: 2 - DNA, 22 bp, Artificial
  Reverse Primer
caccctcatctatctcccaact

SEQ ID NO: 3 - DNA, 21 bp, Artificial
  Forward Primer
cttcacacccattaggatgga

SEQ ID NO: 4 - DNA, 24 bp, Artificial
  Reverse Primer
catttccagcagtgatatatgagg

SEQ ID NO: 5 - DNA, 24 bp, Artificial
  Forward Primer
gagagctcacattctagtcagcct

SEQ ID NO: 6 - DNA, 20 bp, Artificial
  Reverse Primer
atgccaattccccagatgta

SEQ ID NO: 7 - DNA, 20 bp, Artificial
  Forward Primer
gcagcagagcaagaccctat

SEQ ID NO: 8 - DNA, 20 bp, Artificial
  Reverse Primer
atgggtgccttgggtagatt

SEQ ID NO: 9 - DNA, 20 bp, Artificial
  Forward Primer
tctgtctgctgcctcctaca

SEQ ID NO: 10 - DNA, 22 bp, Artificial
  Reverse Primer
gatcctatttttcttggggcta

SEQ ID NO: 11 - DNA, 21 bp, Artificial
  Forward Primer
tgcgcgttaagttaattggtt

SEQ ID NO: 12 - DNA, 20 bp, Artificial
  Reverse Primer
gtaaggtggaaacccccact

SEQ ID NO: 13 - DNA, 21 bp, Artificial
  Forward Primer
cccacctcagaagtcagtgag

SEQ ID NO: 14 - DNA, 20 bp, Artificial
  Reverse Primer
gcaataattctccccaagga

SEQ ID NO: 15 - DNA, 20 bp, Artificial
  Forward Primer
ctgggcaacaagagcaaaat

SEQ ID NO: 16 - DNA, 26 bp, Artificial
  Reverse Primer
tttgttgttgttcattgacttcagtc

SEQ ID NO: 17 - DNA, 20 bp, Artificial
  Forward Primer
cctgggcaataagagctgag

SEQ ID NO: 18 - DNA, 20 bp, Artificial
  Reverse Primer
ccagcccacttttctgttgt

(19) United States
(12) Patent Application Publication
(10) Pub. No.: US 2009/0023138 A1
(43) Pub. Date: Jan. 22, 2009

(54) ORAL CANCER MARKERS AND THEIR DETECTION

(75) Inventors: Douglas D. Burkett, Gilbert, AZ (US); David Sidransky, Baltimore, MD (US); Antonette C.P. Allen, Severn, MD (US); Francis A. Chiafari, Ellicott City, MD (US); Mark Bride, Peoria, AZ (US); Yu Ping Maguire, Kensington, CA (US)

Correspondence Address: JEFFER, MANGELS, BUTLER & MARMARO, LLP, 1900 AVENUE OF THE STARS, 7TH FLOOR, LOS ANGELES, CA, 90067, US

(73) Assignee: ZILA BIOTECHNOLOGY, INC., Phoenix, AZ (US)

(21) Appl. No.: 11/779,236

(22) Filed: Jul. 17, 2007

(51) Int. Cl. C12Q 1/68; G01N 33/574

(52) U.S. Cl. 435/6; 436/64

(57) ABSTRACT

Methods of detecting progression from precancer to cancer are provided utilizing toluidine blue staining as well as detecting allelic variation at microsatellite loci. An allelic variation in one or more locus is indicative of a progression from precancer to cancer.

### FIELD OF INVENTION

The present invention relates generally to the detection of the loss of oral cancer chromosomal loci, and to the detection of microsatellite DNA sequence mutations in markers associated with oral cancer.

### BACKGROUND OF THE INVENTION

Oral cancer is the sixth most common lethal malignancy worldwide. Therefore, the early diagnosis of oral cancer is very important to survival. When identified at an early stage, oral cancers have about an 80-90% survival rate. Unfortunately, at this time, the majority of cases are found as late stage cancers, and this accounts for the very high death rate.

One of the best approaches to identifying genetic changes critical to oral cancer progression is to compare progressing and nonprogressing oral premalignant lesions. A central dogma of carcinogenesis is that alteration in critical control genes underlies malignant transformation. Progressing lesions are genetically different from their morphologically similar nonprogressing counterparts. The development of a test with sufficient specificity and sensitivity for detection of such differences would be useful in predicting the behavior of oral lesions. As a result, clinicians would be able to identify which patients with histologically benign and/or low-grade lesions should be managed more aggressively, either by frequent screening or by early treatment, using traditional approaches such as surgery, or newer techniques such as chemopreventive regimes.

### BRIEF SUMMARY OF PREFERRED EMBODIMENTS

The present invention is directed to methods and kits for the early detection of progression to oral cancer.

The following is a brief summary of the preferred embodiments of the invention.

One preferred embodiment of the invention is a method for detecting cancer or precancer in a subject, the method comprising: determining a first ratio of a level of microsatellite DNA present at a first allele to a level of microsatellite DNA present at a second allele in an oral epithelial cell of the subject; determining a second ratio of a level of microsatellite DNA present at a first allele to a level of microsatellite DNA present at a second allele in a nonepithelial cell of the subject; comparing the first ratio to the second ratio; wherein the subject is heterozygous for the genetic locus; wherein the first and second alleles of both the oral epithelial cells and the nonepithelial cells are at the genetic locus, and the genetic locus comprises microsatellite DNA; wherein the genetic locus is at least one of D3S3597, D3S1067, D3S1300, D3S4103, D9S171, IFN-A, D9S1748, D17S695, or tp53; and wherein a difference between the first and second ratios is indicative of cancer or precancer. This may be known as loss of heterozygosity (LOH) and/or microsatellite instability (MIA). In certain embodiments, the level of microsatellite DNA present at the first and/or the second allele of the oral epithelial cell is lower than the level of microsatellite DNA present at the first and/or second allele of the nonepithelial cell. For example, the level of microsatellite DNA present at the first allele of the oral epithelial cell may be lower than the level of microsatellite DNA present at the first allele of the nonepithelial cell. Preferably, the method is used for the prognosis and/or diagnosis of oral cancer in the subject. The method may be used to determine whether an oral lesion will become cancerous.

Another preferred embodiment of the invention is a method of analyzing microsatellite loci, the method comprising: (a) extracting DNA from paraffin embedded samples of an oral epithelial cell from a subject and of a nonepithelial cell from the subject; (b) providing primers for amplifying a first and a second allele in the oral epithelial cell at a microsatellite locus and a first and a second allele in the nonepithelial cell at the microsatellite locus; (c) amplifying the microsatellite locus; wherein the microsatellite locus comprises at least one of D3S3597, D3S1067, D3S1300, D3S4103, D9S171, IFN-A, D9S1748, D17S695, or tp53; (d) determining a first ratio of a level of microsatellite DNA present at the first allele to level of microsatellite DNA present at the second allele in the oral epithelial cell; determining a second ratio of a level of microsatellite DNA present at the first allele and a level of microsatellite DNA present at the second allele in the nonepithelial cell; and (e) comparing the first and the second ratios. Typically, the subject is a heterozygous individual.

Yet another preferred embodiment of the invention is a method of detecting cancer or precancer in a subject, the method comprising: (a) administering a toluidine blue O stain; (b) providing a control sample DNA and a test sample DNA; (c) amplifying at least one microsatellite locus selected from the group consisting of: D3S3597, D3S1067, D3S1300, D3S4103, D9S171, IFN-A, D9S1748, D17S695, and tp53 in the control sample DNA and the test sample DNA; and (d) determining a microsatellite allele ratio of the control sample DNA and a microsatellite allele ratio of the test sample DNA; (e) detecting a difference between the microsatellite allele ratio of the test sample DNA and the microsatellite allele ratio of the control sample DNA.

Other objects, features and advantages will become apparent to those skilled in the art from the following detailed description (e.g., determining loss of heterozygosity by Single Nucleotide Polymorphism (SNP) assays). It is to be understood, however, that the detailed description and specific examples, while indicating exemplary embodiments, are given by way of illustration and not limitation. Many changes and modifications within the scope of the following description may be made without departing from the spirit thereof, and the description should be understood to include all such variations.

### DETAILED DESCRIPTION OF THE PREFERRED EMBODIMENTS

### Definitions

“DNA mutation,” “allelic imbalance,” “allelic instability,” “DNA deletion” or “allelic variation” may refer to a loss of heterozygousity (“LOH”) and/or microsatellite instability (“MI”). MI refers to the expansion or contraction of short nucleotide repeats (microsatellites) within a tumor when compared to normal tissue. MI may be accompanied by LOH as described below. The instability/imbalance may be detected as an addition and/or amplification of nucleotide repeats in the DNA. This refers to a mutation in the sequence of the microsatellite DNA wherein the resulting microsatellite DNA sequence has more DNA repeats than the sequence found in normal (non-tumor) cells. The instability may be detected as a deletion or addition of nucleotide repeats in the DNA. This refers to a mutation in the sequence of the microsatellite DNA wherein the resulting microsatellite DNA sequence has fewer DNA repeats than the sequence found in normal (non-tumor) cells.

“Markers” or “biomarkers,” as used herein, may be microsatellite markers, microsatellites, short tandem repeats, cancer markers, apoptosis markers, angiogenesis markers, genes, gene products, fragments of markers, markers having covalent modifications, or the like. “Markers,” as used herein, may refer to a locus on a chromosome, and may refer to a fragment of genomic DNA which includes a microsatellite repeat and/or nucleic acid sequences flanking the repeat region.

“Loss of heterozygosity” (“LOH”), as used herein, is a loss of one or both alleles. It may refer to the loss of one or both alleles on one chromosome detected by assaying for markers for which an individual is constitutionally heterozygous. Specifically, LOH may be observed upon amplification of two different samples of genomic DNA from a particular subject, one sample originating from cells from normal biological material, and the other originating from cells from a tumor or from pre-cancerous tissues or cells suspected of having a tumor. The tumor may exhibit LOH if DNA from the normal biological material produces amplified alleles of one ratio and the tumor samples produce a ratio that is substantially different due to reduction or loss of the one allele (LOH) at the same locus.

“Microsatellite ratio,” “ratio,” or “microsatellite allelic ratio” as used herein refers to a ratio of the level of DNA at a first allele to the level of DNA at a second allele at a particular genetic locus or a particular microsatellite locus. As one skilled in the art would appreciate, in LOH cases for example, a first and/or second allele may not be present at the particular genetic locus.

As used herein, a “biological sample” or “DNA sample” may refer to a tissue, cellular, or fluid sample obtained from an individual. The fluid samples may be physiological fluids such as lymph, bile, serum, plasma, urine, synovial fluid, blood, CSF, mucus membrane secretions, or other physiological samples such as stool. Preferably, the biological sample is a cell and/or tissue sample obtained from an oral cancer lesion, or from any other upper aerodigestive tract cancer as referenced below.

As used herein, “stain,” “TBO stain,” “toluidine blue,” or “toluidine blue O” refers to a dye that stains tissue that may transform into, or already has transformed into a malignant phenotype, blue. Other stains with similar function may be substituted for the TBO stain. “Toulidine blue” as used herein may also comprise compositions as disclosed in U.S. Pat. Nos. 6,194,573; 6,086,852; 5,882,627; and 5,372,801; the contents of each of which are herein incorporated by reference.

The terms “cancer,” “cancerous,” “oral cancer,” and “malignant” may refer to or describe the physiological condition in mammals that is typically characterized by unregulated cell growth. These terms may refer to a neoplasm, tumor, oral cancer, oral carcinoma, epithelial dysplasia, head and neck dysplasias and carcinoma, dysplasia in mucosa adjacent to head and neck carcinoma, laryngeal carcinoma, primary squamous cell carcinoma, or premalignant lesions. More particular examples of such cancers include squamous cell cancer, small-cell lung cancer, non-small cell lung cancer, gastrointestinal cancer, bladder cancer, and various types of head and neck cancer. In addition, the terms “cancer,” “cancerous,” “oral cancer,” and “malignant” may refer to upper aerodigestive tract (UADT) cancers, such as, but not limited to, cancers of the lip, tongue, gingivae, buccal mucosa, floor of the mouth, tonsils, oropharynx, nasopharynx, hypopharynx, and larynx.

“Premalignant lesions” or “precancerous lesions” refer to morphologically altered tissue, generalized tissue, or generalized states in which cancer is more likely to occur than in its apparently normal counterpart. Precancerous lesions may be a leukoplakia, erythroplakia, or an erythroleukoplakia.

“Test sample DNA” may refer to any DNA purported to be cancerous or precancerous, such as DNA from dysplastic tissue. This includes, but is not limited to, oral epithelial cells. “Control sample DNA” may refer to noncancerous cells, such as noncancerous epithelial and nonepithelial cells. It may also refer to cells that have not yet exhibited a malignant phenotype or cells that do not stain blue in the presence of TBO. The nonepithelial cells comprise stromal cells, lymphocytes, or other noncancerous cells. “Oral epithelial cell” may refer to any cell of the upper aerodigestive tract. “Oral epithelial cell” as used herein may also refer to any cell purported to be cancerous or precancerous and/or any cell that stains blue with the TBO stain.

As used herein, “change” in microsatellite allelic ratios may refer to a significant change in allelic ratios and/or a difference in allele signal intensities, as is discussed herein and as is determined by the cut-off values.

The following describes methods to identify precancerous/cancerous tissue.

### 1. Toluidine Blue Staining

Precancerous and/or cancerous tissue may be identified by use of selective in vivo staining techniques known in the art such as toluidine blue O (“TBO”) and other cationic supravital marking agents to selectively locate cancerous and precancerous tissue as described in U.S. Published Patent Application Nos. 20040235067; 20050014145; and 20040146919; U.S. Pat. Nos. 4,321,251; 5,372,801; 6,086,852; 6,194,573; and 5,882,627; and Guo et al, Clinical Cancer Research Vol. 7, 1963-1968, the contents of each of which are herein incorporated by reference. TBO is available from, for example, Sigma-Aldrich Corporation and from Zila. TBO has been used for the early detection of oral cancer lesions. Malignant/pre-malignant lesions in the oral cavity will stain “blue” when rinsed with the stain. Tissue scrapes or biopsies may be obtained from these lesions. Cells and/or tissue that stain blue may be further tested for the presence of one or more of the nine markers described herein. Preferably, two oral biopsies, one from a TBO-staining positive area and another from a negative area adjacent to the stain, may be collected and further tested according to the methods of the invention.

Stained lesions may be detected in a subject as follows. A visual oral examination is performed to identify the lesions first. The subject then rinses the oral cavity with approximately 15 ml of a pre-rinse solution, with water for about 20 sec., and with about 30 ml of the TBO solution for about one minute. Lesions may stain blue if malignant/premalignant.

### 2. Selection of Loci to be Amplified or Co-Amplified

The specific nine loci/markers of the invention disclosed herein may be selected by a variety of methods known in the art. A larger number of markers, i.e., greater than nine, may be initially screened. DNA from patients known to have cancer may be screened for alterations in these larger panel of markers. Markers that are altered/mutated in the most cancer samples may be selected, as disclosed in Rosin, Clinical Cancer Research, Vol. 6, 357-362, February 2000, the entire contents of which are herein incorporated by reference.

### 3. Sources of Genomic DNA and Methods of Extraction

Genomic DNA may be extracted from a variety of sources as is known in the art. Genomic DNA may be extracted, for example, from biological samples, paraffin embedded tissue, formalin-fixed paraffin embedded tissue, fresh/frozen tumor/aspirate samples, dry buccal swabs, whole blood, white blood cell pellets, urine, saliva, sputum, bile, stool, cervical tissue, tears, cerebral spinal fluid, serum, plasma, lymphocytes, cell lines, or the like.

Genomic DNA may be extracted using a variety of methods known in the art. DNA may be extracted using commercial kits such as the QIAamp 96 DNA Blood Kit or the BioRobot EZ1 (both available from Qiagen), guanidine-based methods and/or organic extraction, or any other method of DNA extraction known in the art, such as is described in U.S. Pat. No. 6,974,706, the contents of which are herein incorporated by reference.

Extracted DNA samples may be further processed as is known in the art. Sample preparation and separation may involve any of the following procedures, depending on the type of sample collected and/or types of biological molecules searched: concentration, dilution, or adjustment of pH.

### 4. Quantification of DNA

DNA may be quantified by a variety of methods known in the art. For example, gel-based quantification, Pico Green-based methods, Quantifiler™ (available from Applied BioSystems, Inc.) or similar amplification assays, or any other method known in the art may be used. Pico Green-based methods allow for quantification of small quantities of human DNA using fluorescent detection methods. This allows for determination of whether the isolated DNA is suitable for analysis and to adjust the amount of DNA template used in amplification reactions or other procedures.

### 5. Methods of the Amplification and Detection

Any method known in the art for PCR and/or genotyping may be used to practice the invention, such as methods geared to accomplish optimal sensitivity and specificity. The markers disclosed herein may be detected using polymerase chain reaction methods, such as standard PCR, quantitative PCR, real-time PCR, SNPs, RFLP, or any other method known in the art, such as those described for example in U.S. Pat. Nos. 5,210,015; 5,804,375; 5,487,972; 6,174,670; 4,683,202; and 4,683,195; the contents of each of which are herein incorporated by reference. PCR may be manual or automated.

Alternately, the biological sample may be tested to determine the levels of multiple oral cancer biomarkers in a single reaction using an assay capable of measuring the individual levels of different oral cancer biomarkers in a single reaction, such as an array-type assay or assay utilizing multiplexed detection technology (e.g., an assay utilizing detection reagents labeled with different fluorescent dye markers). Multiplex PCR is an example of such an assay. Multiplex PCR involves different primer pairs in the same amplification reaction. Each amplification reaction may contain two or more primer pairs for detection of two or more markers. Preferably, each reaction tube may contain two to five primer pairs for detection of two to five markers. One primer in each primer pair may be fluorescently labeled with fluorecscein, JOE, NED, or the like (see, e.g., Table 2).

Markers may be amplified in vitro, using PCR or the like. Markers may also be cloned in vivo, using cloning techniques known in the art. For example, the nucleic acid (e.g., genomic DNA) may be inserted into a replicable vector for cloning. The vector may, for example, be in the form of a plasmid. The appropriate nucleic acid sequence may be inserted into the vector by a variety of procedures known in the art. Cloning vectors may contain a nucleic acid sequence that enables the vector to replicate in host cells for vector production and amplification.

One or more of the markers thereof may be detected by methods known in the art for detecting DNA. These methods may include microarray, Southern blots, or the like

In addition, the microarray may be spotted with sequences complementary, for example, to the MI and/or LOH-exhibiting markers or other SNP markers from the same locus, as well as with sequences complementary to these markers from normal cells/tissues. Samples may be tested to determine whether they hybridize to the MI and/or LOH-exhibiting markers, or whether they hybridize to the markers from normal cells/tissues and to what extent. DNA samples that have a greater level of hybridization to the MI and/or LOH-exhibiting markers may be deemed to be cancerous or precancerous, and may need to be further characterized.

### 6. Polynucleotide Size Determination

Size of DNA amplified in the foregoing may be determined by many methods known in the art such as gel electrophoresis and capillary electrophoresis.

The methods described herein may be implemented using any device capable of implementing the methods to measure DNA size and quantity as well the ratio of amplified alleles. Examples of devices that may be used, include, but are not limited to, electronic computational devices.

The methods for characterizing the oral cancer lesion may, of course, depend on the format of the assay, the sensitivity/specificity required, and the preference of the practitioner. The invention is not limited by the methods disclosed herein.

The invention also provides kits for carrying out any of the methods described herein. Kits of the invention may comprise reagent(s) for amplifying one or more markers such as the nine markers disclosed herein and/or other markers as is known in the art, and may further include instructions for carrying out a method described herein. These reagents may be, but are not limited to, SEQ. ID NOS. 1-18 (see, e.g., Table 1) as disclosed herein. Kits may also comprise biomarker reference samples, that is, samples from normal tissue useful as reference value. Kits may comprise reagents for detecting marker DNA. In addition, kits may comprise a TBO stain, or the like. Instructions supplied in the kits of the invention are typically written instructions on a label or package insert (e.g., a paper sheet included in the kit), but machine-readable instructions (e.g., instructions carried on a magnetic or optical storage disk) are also acceptable.

One preferred embodiment of the invention is a method for detecting cancer or precancer in a subject, the method comprising: detecting in a test sample DNA of the subject a microsatellite instability at a genetic locus, by determining and comparing a level of microsatellite DNA present at a first allele to a level of microsatellite DNA present at a second allele in an oral cancer cell and a nonepithelial cell; wherein the subject is heterozygous for the genetic locus; wherein the first and second alleles are at the genetic locus; wherein the genetic locus is at least one of the genetic loci selected from the group consisting of D3S1067, D3S3597, D3S1300, D3S4103, D9S171, IFN-A, D9S1748, D17S695, or tp53; and wherein detection of microsatellite instability is indicative of cancer or precancer. Preferably, the method is used to determine of the oral lesion will become cancerous. The method may also be used to diagnose oral cancer in the subject.

In certain embodiments, the first or second allele may not be present at a particular microsatellite locus in the test sample DNA. The first and second alleles, however, may be present, with no allelic losses or additional alleles, in the control sample DNA at the particular microsatellite locus. In addition, as those skilled in the art will appreciate, the first and/or second allele may not necessarily be present, for example in cases of LOH. In certain embodiments, the individual may be homozygous for the particular genetic locus.

Another preferred embodiment of the invention is a method of analyzing microsatellite loci, the method comprising: a) extracting DNA from paraffin embedded samples; b) providing primers for amplifying a microsatellite locus; c) amplifying the microsatellite locus; and (d) determining the size of a DNA fragment produced from said amplification; wherein the microsatellite locus is at least one selected from the group consisting of D3S3597, D3S1067, D3S1300, D3S4103, D9S171, IFN-A, D9S1748, D17S695, or tp53.

Another preferred embodiment of the invention is a method of analyzing a DNA mutation, the method comprising: providing a control sample DNA and a test sample DNA, amplifying at least one genetic locus selected from the group consisting of D3S3597, D3S1067, D3S1300, D3S4103, D9S171, IFN-A, D9S1748, D17S695, and tp53 in the control sample DNA and the test sample DNA, and detecting a difference in fragment size between the test sample DNA and the control sample DNA.

Another preferred embodiment of the invention is a method of detecting cancer or precancer in a subject, the method comprising: determining a size of a DNA fragment amplified at a minimum of one locus selected from the group consisting of D3S3597, D3S1067, D3S1300, D3S4103, D9S171, IFN-A, D9S1748, D17S695, and tp53 using SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15, SEQ. ID NO. 16, SEQ. ID NO. 17, or SEQ. ID NO. 18 (see, e.g., Table 1) as primers in a non-cancerous biological sample, and comparing said size to a size of a DNA fragment at the same locus or loci in a cancerous biological sample, wherein a difference in size is indicative of microsatellite instability.

Yet another preferred embodiment of the invention is a method of analyzing microsatellite loci, the method comprising providing primers for amplifying a set of at least two microsatellite loci of human DNA, wherein the set of at least two microsatellite loci are selected from the group consisting of D3S1067, D3S3597, D3S1300, D3S4103, D9S171, IFN-A, D9S1748, D17S695, and tp53; amplifying the set of at least two microsatellite loci from at least one sample of genomic DNA in a multiplex amplification reaction, using the primers, thereby producing amplified DNA fragments; and determining the size of the amplified DNA fragments. In one embodiment of the method, the set of at least two microsatellite loci of human DNA is a set of at least three microsatellite loci of human DNA. In another embodiment of the method, the set of at least three microsatellite loci of human DNA is a set of at least four microsatellite loci of human DNA. In yet another embodiment of the method, the set of at least four microsatellite loci of human DNA is a set of at least five microsatellite loci of human DNA. Preferably, the primers have a nucleic acid sequence selected from the group of primer sequences identified by SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15, SEQ. ID NO. 16, SEQ. ID NO. 17, and SEQ. ID NO. 18 as shown in Table 1. The microsatellite loci may be co-amplified using one primer for each locus which is fluorescently labeled and one primer for each locus that is unlabeled. The sample of genomic DNA comprises a first sample of genomic DNA originating from normal non-cancerous biological material from an individual, and a second sample of genomic DNA originating from a tumor and/or precancerous material of the individual. The method may further comprise correlating microsatellite instability results with the prognosis and/or diagnosis of oral cancer. The method may comprise extracting DNA from paraffin embedded tissue.

Another preferred embodiment of the invention is a method of detecting cancer or precancer in a subject, the method comprising: (a) administering a toluidine blue O stain; (b) providing a control sample DNA and a test sample DNA; (c) amplifying at least one of D3S3597, D3S1067, D3S1300, D3S4103, D9S171, IFN-A, D9S1748, D17S695, and tp53 in the control sample DNA and the test sample DNA, and (d) detecting a significant change (i.e., a difference between the microsatellite allele ratio of the test sample DNA and the microsatellite allele ratio of the control sample DNA) in allele signal intensity ratios of the control sample DNA and the test sample DNA, wherein (b), (c), and (d) is used to verify (a).

Yet another preferred embodiment of the invention is a kit for detecting a DNA mutation, the kit comprising oligonucleotide primers that are complementary to a nucleotide sequence that flanks nucleotide repeats of microsatellite DNA, wherein the nucleotide repeats of microsatellite DNA comprise at least one of D3S3597, D3S1067, D3S1300, D3S4103, D9S171, IFN-A, D9S1748, D17S695, and tp53. Preferably, the kit comprises D3S3597, D3S1067, D3S1300, D3S4103, D9S171, IFN-A, D9S1748, D17S695, and tp53. The kit may further comprise a detectably labeled deoxyribonucleotide. Preferably, the kit may contain a stain for detecting cancerous oral lesions. More preferably, the stain is TBO.

Another preferred embodiment of the invention is a method of detecting cancer or precancer in a subject, the method comprising: (a) administering a toluidine blue O stain; (b) detecting allelic variation in at least one locus selected from the group consisting of D3S3597, D3S1067, D3S1300, D3S4103, D9S171, IFN-A, D9S1748, D17S695, and tp53. Preferably, the results of (b) are used to verify the results of (a). Most preferably, a positive TBO stain, i.e., a stain indicative of oral cancer, will exhibit allelic variation in least one of the loci.

Another embodiment of the invention comprises a method of analyzing microsatellite loci, the method comprising detecting allelic variation at a genetic locus comprising D3S3597, D3S1067, D3S1300, D3S4103, D9S171, IFN-A, D9S1748, D17S695, and tp53.

### EXAMPLES

The following examples are provided to illustrate the invention, but are not intended to limit the scope of the invention in any way.

### Example 1

DNA was isolated from whole blood or white blood cell pellets using the QIAamp 96 DNA Blood kit, available from Qiagen. DNA was also isolated from fresh/frozen tissue/aspirate using generally guanidine-based methods and following the steps of protein digestion, phenol/chloroform extraction, DNA precipitation, and concentration, as is known in the art.

Tissue was processed for paraffin embedding according to standard laboratory procedures and was fixed with 10% neutral buffered formalin. Oral mucosal specimens were embedded on edge. Tissue was sectioned using standard microtomy procedures, placed upon microscope slides, and stained with H & E. The pathology of these tissues was reviewed and the slides marked for the presence of stromal or non-epithelial and epithelial tissue.

In order to extract DNA from the paraffin embedded samples, the samples were scraped from the slides, de-paraffinized and re-hydrated as is known in the art. Subsequently, the steps of sample protein digestion, DNA extraction, and re-suspension was completed in order to isolate the DNA from the paraffin-embedded samples. Alternatively, DNA was extracted from paraffin embedded tissue sections using the BioRobot EZ1 DNA system (available from Qiagen), according to the manufacturer's protocol.

DNA was also extracted from dry buccal swabs using standard organic extraction techniques, as is known in the art. Total DNA (genomic and mitochondrial) was isolated from buccal cells using the EZ1 DNA Tissue Kit (available from Qiagen), in combination with the BioRobot EZ1 workstation (available from Qiagen), according to the manufacturer's protocol and standard techniques known in the art.

### Example 2

DNA was subsequently quantified using any method known in the art, for example gel-based DNA quantification, Pico Green quantification, and the Quantifiler™ assay. Pico Green quantification was performed using the Pico Green ds DNA Quantification reagent and the TH01 GenePrint STR System, both available from Promega. Assays were performed according to the manufacturer's protocol. AmpliTaq Gold Polymerase and Gold ST*R 10X Buffer are available from Applied Biosystems, Inc. or Roche. Standard used was Human DNA Standard 9947A. Plates were scanned on a Hitachi FMBIOII or were read in a fluorimeter. The Quantifiler™ assay was performed using ABI Prism 7900HT Sequence Detection System (available from Applied Biosystems, Inc.) and ABI Quantifiler Human DNA Quantification Kit (available from Applied Biosystems, Inc.). The Quantifiler™ amplification was performed according to the manufacturer's protocol.

### Example 3

PCR reactions (STR amplifications) were performed using isolated DNA as follows. Each primer/oligonucleotide marker was identified by locus name. Oligonucleotide markers included D3S1067, D3S3597, D3S4103, D9S171, IFN-A, D9S1748, D17S695, tp53, and D3S1300 and were obtained from a certified oligonucleotide manufacturer. The dinucleotide loci were D3S1067, D3S3597, D3S1300, D9S171, IFN-A, and D9S1748. The trinucleotide locus was D3S4103; tetranucleotide locus was D17S695, and the pentanucleotide locus was tp53. Primers used included the following: SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15, SEQ. ID NO. 16, SEQ. ID NO. 17, and SEQ. ID NO. 18. One primer from each set (forward/reverse) was end-labeled with a fluorescent probe such as 5FAM, JOE, or NED (see, e.g., Table 1).

Each reaction sample had 1X of 10X Hi Fi Buffer available from Invitrogen, 200 μM of 25 μM dNTPS, 1.5 mM of 50 mM MgSO₄, and 2 U of 5 U/μl Taq Platinum Hi-Fi from Invitrogen. The final concentrations of the forward and reverse primers ranged from 0.18-1.00 pmol/reaction; they were added to the foregoing mix. Sterile MBG H₂O was added to bring the volume to 23 μl. 1 μl BSA was added directly to each sample. Each reaction contained 1 ng of DNA (see, e.g., Table 2).

DNA was subjected to one of two amplification cycling parameters as determined by the multiplex primer set: a) 33 cycles of amplification in a thermocyler as follows: 95° C. for 11 min. (1 cycle); 94° C. for 30 sec., 60.1° C. for 30 sec., 70° C. for 45 sec. (30 cycles); 60° C. for 30 min. (1 cycle); and a final hold at 4° C. (1 cycle). b) DNA was subjected to 33 cycles of amplification in a thermocycler as follows: 95° C. for 11 min (1 cycle); 94° C. for 30 sec., 61.7° C. for 30 sec., 70° C. for 45 sec. (30 cycles); 60° C. for 30 min. (1 cycle); and a final hold at 4° C. A 1 ng/μl dilution of each sample was produced. The positive control, 9947A, obtained from the manufacturer at 10 ng/μL, was diluted to the concentration of 1 ng/μL for use. The negative control was MBG H₂O.

Amplification reactions were set-up on 96-well plates as follows. 1 μl of each diluted DNA sample was transferred to appropriate wells in the amplification plates. PCR “master mix” or “cocktail” was added to each of the reaction samples as is known in the art. Positive controls, blank, and negative isolation reagent controls were added as necessary. The reaction plate was placed in a Techne or a PE thermocycler, selecting the appropriate program for the primer set being amplified.

### Example 4

Capillary electrophoresis was conducted on ABI Prism 3100-Avant (available from Applied Biosystems, Inc.) according to the manufacturer's protocol. Software used was ABI Prism 3100-Avant Data Collection Software, version 1.0 or higher, and GeneMapperID Software, version 3.2 or higher. The capillary electrophoresis produced the electronic data for the final analysis. Size standards used were Genescan-500 (ROX) (available from Applied Biosystems, Inc.).

### Example 5

Allelic ladder sizing standards for custom primer sets were prepared. Amplified samples were chosen for further processing and were used as individual samples or as “mixed sample” lanes.

### Example 6

Following the 3100 Avant run and analysis, GeneMapperID software (preferably v 3.2 or higher, available from Applied Biosystems, Inc.) was used to analyze DNA fragments according to manufacturer's protocol. The software was used to facilitate the analysis of STR data images created by capillary electrophoresis of amplified DNA fragments on the ABI 3100 Avant Genetic Analyzer. Fragment size was automatically assigned based on an internal size standard that was co-electrophoresed with each sample. Alleles were assigned based on comparison of the fragment size of the unknown peak to that of the allelic ladder.

GeneMapperID software v. 3.2 or higher from Applied Biosystems, Inc. was then used to convert allele sizes into allele designations automatically according to the manufacturer's protocol. The results from the ABI 3100 Avant were imported and filtered by algorithms in GeneMapperID software v. 3.2 to provide final results such as allele calls and automated table building. Genotypes were assigned by comparing the sizes obtained for unknown samples to the sizes obtained for the alleles in the allelic ladder. Off ladder alleles were then determined.

### Example 7

Alleles were designated in accordance with the recommendations of the DNA Commission of the International Society for Forensic Genetics (ISFG).

The threshold values for the multiplexes were: a) 4-plex: the threshold level for the blue channel was set at 242 RFU and for the green channel, 203 RFU; these thresholds were set at 2 standard deviation; b) 5-plex: the threshold level for the blue channel was set at 201 RFU; for the green channel, 245 RFU; and for the yellow channel, 174 RFU; these thresholds were set at 2 standard deviations. Thresholds were also calculated for stutter and minus A peaks. Alleles, with values lower than the threshold setting, were not called by the automated software; these samples were repeated. The RFU values were set locus-specifically. Target values for the loci were: D17S695, D9S171, D3S1300, and D3S3597: 2000±500 RFU; D9S1748, D3S4103, and tp53: 1500±500 RFU; D3S1067, and IFN-A: 1000±500 RFU. All peaks with a height greater than 174 RFU present in the size range of the system were examined. Any peak with a height less than 174 RFU was not reported.

A “positive case” consisted of the following: loss of heterozygosity (LOH)—alleles detected in two different samples did not match when they were assigned different allele designations. A case was deemed LOH when the obligate allele(s) possessed by the normal stroma were imbalanced beyond the cut-off values in dyplastic tissue, and no new additional allele(s) were assigned. Instability—alleles detected in two different samples did not match when they were assigned different allele designations. A case was designated MI when additional (new) allele(s) were assigned to the dysplastic tissue sample exclusively.

Cut-Off Chart

| Locus | Lower Limit | Upper Limit |
|---|---|---|
| D9S171 | 0.44 | 2.28 |
| D17S695 | 0.52 | 1.89 |
| D3S1067 | 0.48 | 2.10 |
| D3S3597 | 0.53 | 1.89 |
| D9S1748 | 0.54 | 1.87 |
| D3S4103 | 0.47 | 2.13 |
| IFN-A | 0.38 | 2.62 |
| tp53 | 0.49 | 2.05 |
| D3S1300 | 0.38 | 2.63 |

Interpretation of the STR profiles took into account “pull-up peaks”, stutter, lack of 3′ addition, shoulder peaks, and off ladder alleles.

Identical alleles were determined by the following: a) two samples were assigned the same allele designation based on corrected size calculations. The interpretation of the proper allele designation was assisted by software, but made final by reader. Virtual co-migration in different runs, after correction of size estimations from internal standards, represented the preferred method of DNA profile matching in the lab. Allele assignments were performed precisely enough such that samples were compared from different runs. Allele designations were portable across all runs, and samples matched at all loci tested.

All publications and patent applications cited above are incorporated by reference in their entirety for all purposes to the same extent as if each individual publication or patent application were specifically and individually indicated to be incorporated by reference. Although the present invention has been described in some detail by way of illustration and example for purposes of clarity and understanding, it will be apparent that certain changes and modifications may be practiced within the scope of the appended claims.

TABLE 1
Nucleic Acid Primer Sequences

(SEQ ID NO.: 1)

| | |
|---|---|
| D3S1067 F | 5′ CCC CAG ATT TTG AGC ACT ACC 3′ |

(SEQ ID NO.: 2)

| | |
|---|---|
| D3S1067 R | 5′ CAC CCT CAT CTA TCT CCC AAC T 3′ |

(SEQ ID NO.: 3)

| | |
|---|---|
| D3S3597 F | 5′ CTT CAC ACC CAT TAG GAT GGA 3′ |

(SEQ ID NO.: 4)

| | |
|---|---|
| D3S3597 R | 5′ CAT TTC CAG CAG TGA TAT ATG AGG 3′ |

(SEQ ID NO.: 5)

| | |
|---|---|
| D3S1300 F | 5′ GAG AGC TCA CAT TCT AGT CAG CCT 3′ |

(SEQ ID NO.: 6)

| | |
|---|---|
| D3S1300 R | 5′ ATG CCA ATT CCC CAG ATG TA 3′ |

(SEQ ID NO.: 7)

| | |
|---|---|
| D3S4103 F | 5′ GCA GCA GAG CAA GAC CCT AT 3′ |

(SEQ ID NO.: 8)

| | |
|---|---|
| D354103 R | 5′ ATG GGT GCC TTG GGT AGA TT 3′ |

(SEQ ID NO.: 9)

| | |
|---|---|
| D9S171 F | 5′ TCT GTC TGC TGC CTC CTA CA 3′ |

(SEQ ID NO.: 10)

| | |
|---|---|
| D9S171 R | 5′ GAT CCT ATT TTT CTT GGG GCT A 3′ |

(SEQ ID NO.: 11)

| | |
|---|---|
| IFN-A F | 5′ TGC GCG TTA AGT TAA TTG GTT 3′ |

(SEQ ID NO.: 12)

| | |
|---|---|
| IFN-A R | 5′ GTA AGG TGG AAA CCC CCA CT 3′ |

(SEQ ID NO.: 13)

| | |
|---|---|
| D9S1748 F | 5′ CCC ACC TCA GAA GTC AGT GAG 3′ |

(SEQ ID NO.: 14)

| | |
|---|---|
| D9S1748 R | 5′ GCA ATA ATT CTC CCC AAG GA 3′ |

(SEQ ID NO.: 15)

| | |
|---|---|
| D17S695 F | 5′ CTG GGC AAC AAG AGC AAA AT 3′ |

(SEQ ID NO.: 16)

| | |
|---|---|
| D17S695 R | 5′ TTT GTT GTT GTT CAT TGA CTT CAG TC 3′ |

(SEQ ID NO.: 17)

| | |
|---|---|
| tp53 F | 5′ CCT GGG CAA TAA GAG CTG AG 3′ |

(SEQ ID NO.: 18)

| | |
|---|---|
| tp53 R | 5′ CCA GCC CAC TTT TCT GTT GT 3′ |

TABLE 2
PCR conditions for Multiplex

| | | Final conc. | Primer Set | Amplification | |
|---|---|---|---|---|---|
| Locus | Primer Name | (pmol/rxn) | in Plex | Template | Fluor |
| D9S171 | D9S171 Forward | 0.25 | D17S695 | 1 | Fluorecscein |
| | D9S171 Reverse | | D3S1067 | | |
| | | | D3S3597 | | |
| | | | D9S1748 | | |
| D17S695 | D17S695 Forward | 0.70 | D9S171 | 1 | Fluorecscein |
| | D17S695 Reverse | | D3S1067 | | |
| | | | D3S3597 | | |
| | | | D9S1748 | | |
| D3S1067 | D3S1067 Forward | 0.50 | D9S171 | 1 | JOE |
| | D3S1067 Reverse | | D17S695 | | |
| | | | D3S3597 | | |
| | | | D9S1748 | | |
| D3S3597 | D3S3597 Forward | 1.00 | D9S171 | 1 | JOE |
| | D3S3597 Reverse | | D17S695 | | |
| | | | D3S1067 | | |
| | | | D9S1748 | | |
| D9S1748 | D9S1748 Forward | 1.00 | D9S171 | 1 | NED |
| | D9S1748 Reverse | | D17S695 | | |
| | | | D3S1067 | | |
| | | | D3S3597 | | |
| D3S4103 | D3S4103 Forward | 0.18 | IFN-A | 2 | Fluorecscein |
| | D3S4103 Reverse | | D3S1300 | | |
| | | | TP53 | | |
| D3S1300 | D3S1300 Forward | 0.32 | D3S4103 | 2 | Fluorecscein |
| | D3S1300 Reverse | | IFN-A | | |
| | | | TP53 | | |
| IFN-A | IFN-A Forward | 0.25 | D3S4103 | 2 | Fluorecscein |
| | IFN-A Reverse | | TP53 | | |
| TP53 | TP53 Forward | 0.65 | D3S4103 | 2 | JOE |
| | TP53 Reverse | | IFN-A | | |
| | | | D3S1300 | | |

What is claimed is:

1. A method for detecting cancer or precancer in a subject, the method comprising:
(a) determining a first ratio of a level of microsatellite DNA present at a first allele to a level of microsatellite DNA present at a second allele in an oral epithelial cell of the subject; determining a second ratio of a level of microsatellite DNA present at a first allele to a level of microsatellite DNA present at a second allele in a nonepithelial cell of the subject; and
(b) comparing the first ratio to the second ratio;
wherein the subject is heterozygous for the genetic locus; wherein the first and second alleles of the oral epithelial cell and the nonepithelial cell are at the genetic locus, and the genetic locus comprises microsatellite DNA; wherein the genetic locus is at least one of D3S3597, D3S1067, D3S1300, D3S4103, D9S171, IFN-A, D9S1748, D17S695, or tp53; and wherein a difference between the first and second ratios is indicative of cancer or precancer.

2. The method of claim 1, wherein the level of microsatellite DNA present at the first allele of the oral epithelial cell is lower than the level of microsatellite DNA present at the first allele of the nonepithelial cell.

3. A method of analyzing a microsatellite locus, the method comprising:
a) extracting DNA from paraffin embedded samples of an oral epithelial cell from a subject and of a nonepithelial cell of the subject;
b) providing primers for amplifying a first and a second allele in the oral epithelial cell at the microsatellite locus and a first and second allele in the nonepithelial cell at the microsatellite locus;
c) amplifying the microsatellite locus, wherein the microsatellite locus comprises at least one of D3S1067, D3S1300, DS4103, D3S3597, D9S171, IFN-A, D9S1748, D17S695, and tp53; and
d) determining a first ratio of a level of microsatellite DNA present at the first allele to a level of microsatellite DNA present at the second allele in the oral epithelial cell, and determining a second ratio of a level of microsatellite DNA present at the first allele and a level of microsatellite DNA present at the second allele in the nonepithelial cell; and
e) comparing the first and second ratios.

4. A method of analyzing microsatellite loci, the method comprising detecting allelic variation at a genetic locus comprising D3S1067, D3S3597, D3S1300, D3S4103, D9S171, IFN-A, D9S1748, D17S695, and tp53.

5. The method of claim 1, wherein the method is used to diagnose oral cancer in the subject.

6. The method of claim 1, wherein the method is used to determine if an oral lesion will become cancerous.

7. A method of detecting cancer or precancer in a subject, the method comprising:
a) determining a size of a DNA fragment amplified at a minimum of one locus selected from the group consisting of D3S3597, D3S1067, D3S1300, D3S4103, D9S171, IFN-A, D9S1748, D17S695, or tp53 using SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15, SEQ. ID NO. 16, SEQ. ID NO. 17, or SEQ. ID NO. 18 as primers in a non-cancerous biological sample and comparing said size to a size of a DNA fragment at the same locus in a cancerous biological sample; wherein a difference in size is indicative of microsatellite instability.

8. A method of analyzing microsatellite loci, the method comprising:
(a) providing primers for amplifying a set of at least two microsatellite loci of human DNA, wherein the set of at least two microsatellite loci are selected from the group consisting of D3S1067, D3S3597, D3S1300, D3S4103, D9S171, IFN-A, D9S1748, D17S695, and tp53;
(b) amplifying the set of at least two microsatellite loci from a sample of genomic DNA in a multiplex amplification reaction, using the primers, thereby producing amplified DNA fragments; and
c) determining the size of amplified DNA fragments.

9. The method of claim 8, wherein the primers provided in step (a) has a nucleic acid sequence selected from the group of primer sequences identified by SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15, SEQ. ID NO. 16, SEQ. ID NO. 17, and SEQ. ID NO. 18.

10. The method of claim 9, wherein the set of at least two microsatellite loci is a set of at least three microsatellite loci.

11. The method of claim 10, wherein the set of at least three microsatellite loci is a set of at least four microsatellite loci.

12. The method of claim 9, wherein the set of at least two microsatellite loci is amplified in step (b) using at least one oligonucleotide primer for each locus which is fluorescently labeled.

13. The method of claim 1, further comprising correlating the microsatellite instability with a prognosis of oral cancer.

14. A method of detecting cancer or precancer in a subject, the method comprising:
(a) administering a toluidine blue O stain;
(b) providing a control sample DNA and a test sample DNA;
(c) amplifying at least one microsatellite locus selected from the group consisting of D3S3597, D3S1067, D3S1300, D3S4103, D9S171, IFN-A, D9S1748, D17S695, and tp53 in the control sample DNA and the test sample DNA; and
(d) determining a microsatellite allele ratio of the control sample DNA and a microsatellite allele ratio of the test sample DNA at the microsatellite locus;
(e) detecting a difference between the microsatellite allele ratio of the test sample DNA and the microsatellite allele ratio of the control sample DNA.

15. The method of claim 14, wherein the difference is a significant difference in the allele intensity ratios of the control sample DNA and the test sample DNA.

16. The method of claim 14, wherein (b), (c), (d), and (e) is used to verify (a).

17. A kit for detecting a DNA mutation, the kit comprising oligonucleotide primers that are complementary to a nucleotide sequence that flanks nucleotide repeats of microsatellite DNA, wherein the nucleotide repeats of microsatellite DNA comprise at least one of D3S3597, D3S1067, D3S1300, D3S4103, D9S171, IFN-A, D9S1748, D17S695, and tp53.

18. The kit of claim 17, further comprising a detectably labeled deoxyribonucleotide.

19. The kit of claim 17, further comprising a stain for detecting cancerous oral lesions.

20. The kit of claim 19, wherein the stain is toluidine blue O.

21. A method of detecting cancer or precancer in a subject, the method comprising: (a) administering a toluidine blue O stain and (b) detecting allelic variation in at least one locus selected from the group comprising D3S3597, D3S1067, D3S1300, D3S4103, D9S171, IFN-A, D9S1748, D17S695, and tp53.

22. The method of claim 21, wherein (b) is used to verify (a).
